SEQUENCE LISTING (3 sequences)

SEQ ID NO: 1 - protein, 51 aa, Homo sapiens
VVSHFNDCPDSHTQFCFHGTCRFLVQEDKPACVCHSGYVGARCEHADLLAV

SEQ ID NO: 2 - DNA, 32 bp, Artificial Sequence
  Synthetic Construct
gaattcgtggtgtccccattttaatgactgcc

SEQ ID NO: 3 - DNA, 25 bp, Artificial Sequence
  Synthetic Construct
gaattctcaggccaggaggtccgca

(19) United States
(12) Patent Application Publication
(10) Pub. No.: US 2022/0177533 A1
(43) Pub. Date: Jun. 9, 2022

(54) Recombinant TGF a for wound healing purposes, and the process thereof

(71) Applicant: SREE CHITRA TIRUNAL INSTITUTE FOR MEDICAL SCIENCES AND TECHNOLOGY, THIRUVANANTHAPURAM (IN)

(72) Inventors: THEKKUVEETTIL Kumar Anoop, THIRUVANANTHAPURAM (IN); Salini BHASKER, THIRUVANANTHAPURAM (IN); Geethu SHAJI, THIRUVANANTHAPURAM (IN)

(73) Assignee: SREE CHITRA TIRUNAL INSTITUTE FOR MEDICAL SCIENCES AND TECHNOLOGY, THIRUVANANTHAPURAM (IN)

(21) Appl. No.: 17/266,898

(22) PCT Filed: Aug. 8, 2019
(86) PCT No.: PCT/IN2019/050578

§ 371 Date: Feb. 8, 2021

(30) Foreign Application Priority Data
Aug. 8, 2018 (IN) 201841029835

(51) Int. Cl. C07K 14/495; C12N 15/70; A61P 17/02

(52) U.S. Cl. CPC C07K 14/495; A61K 38/00; A61P 17/02; C12N 15/70

(57) ABSTRACT

In one aspect of the invention, the present invention discloses and claims a recombinant transforming growth factor alpha (TGF α) consisting of Sequence ID No.: 1, craving out from the whole TGF α, and cloned for the wound healing applications. Further it discloses primers for amplifying TGF α consisting of Sequence ID Nos.: 2 and 3, wherein sequence ID No.: 2 is forward primer, and the sequence ID No.: 3 is reserve primer for TGF α. It further discloses a process of preparation of a vector comprising essentially of Sequence ID No.: 1, said process comprising the steps of multiplying the desired gene fragment through PCR using primers of Sequence ID No.: 2 and 3. Further, a pharmaceutical composition comprising essentially of Sequence ID No.: 1, along with additives, fillers and addendums is disclosed. Finally, it discloses a method of wound healing using the recombinant TGF α comprising application of a pharmaceutical composition.

### FIELD OF THE INVENTION

The present invention relates to a process to Recombinant Transforming Growth Factor Alpha (TGFα) for various wound healing applications and the process for the preparation thereof.

This invention further relates to the synthesis of a recombinant Transforming Growth Factor-alpha (TGFα) in a prokaryotic expression system, which have a potential role in wound healing by reconstructing the epithelial tissues of chronic wounds thus increasing the relative speed of wound healing with minimal scar formation. The active recombinant protein can be harvested at a minimum cost.

### SEQUENCE LISTING

Applicant incorporates by reference a CRF sequence listing having file name SYZ0003PA_SEQ.txt (1.28 kB), created Sep. 7, 2021.

### BACKGROUND AND PRIOR ARTS OF THE PRESENT INVENTION

The complex process of wound healing involves inflammatory response in the wound area, followed by proliferation of wound cells and remodeling. In these pathways growth factors play a critical role in enhanced cellular proliferation resulting in faster wound healing. Growth factors are soluble secreted signaling poly peptides, which binds to their respective receptors present on the cell membrane and signals specific cellular responses. These responses trigger a wide range of cellular actions, including cell survival, and control over migration, differentiation or proliferation of a specific subset of cells.

Transforming Growth Factor Alpha (TGF α) is one such growth factors, which stimulates growth and migration of keratinocytes and fibroblasts in skin wounds. TGF α binds to EGF receptor with high affinity to initiate cellular responses. Besides, TGF α along with cell proliferation can enhance in reducing excessive scar formation as a result of chronic wounds.

It is well established that TGFα has similar role as epidermal growth factor (EGF) by binding to the same receptors. (Ref.: Schultz, Rotatori and Clark: EGF and TGF-alpha in wound healing and repair DOI:10.1002/jcb.240450407).

Transforming Growth Factor-alpha (TGFα) is one of the members of epidermal growth factor (EGF) receptor ligand family. This protein is known to stimulate cell growth and knockout of the gene resembles to that of EGF receptor knockouts with a characteristic “waved coat phenotype” in mouse models. In human, TGFα gene is in short arm of chromosome 2 (2pl3) with six exons, the whole gene spans a 138.7 kb region. The mRNA is of the size 4326 bases coding for a 160 amino acid peptide. 23 amino acids at the amino terminal are removed as the pre-pro TGF α translocate to ER lumen. Further, this pro-TGFα becomes a mature peptide by two metalloprotease cleaves both at distal and proximal sites of EGF domain. The final function peptide is of 62 amino acids.

Growth factors have a critical role in wound healing because the healing process involves wound cell migration and mitosis, extracellular matrix remodeling and neovascularization. There are data suggesting that local production of peptide growth factors have a critical influence in the healing process. During normal wound healing of tissues, especially skin and cornea tissues, both EGF and TGFα have critical roles, facilitated through platelets, keratinocytes and activated macrophages. There are evidences for enhanced healing of a variety of wounds in animals and patients by treatment with TGFα. Tropical application of these growth factors found to accelerate the epidermal regeneration of partial thickness burn wounds on pigs or dermatome wounds on patients.

Publication no WO1996036709A1 (Application no PCT/US1995/006386) relates to Transforming Growth Factor alpha HII. The polypeptide of the TGF-alpha HII has an amino acid sequence homology to human TGF-alpha and it stimulates wound healing, restores normal neurological functioning after trauma or AIDS dementia, treats ocular disorders, stimulates angiogenesis for the treatment of burns, ulcers and corneal incisions and stimulate embryogenesis. This invention provides antibodies against such polypeptides which may be used to inhibit the action of such polypeptides. For e.g.: in the treatment of corneal inflammation, neoplasia and psoriasis. Further, it also provides diagnostic assays for detecting diseases related to over expression of the polypeptide and mutations in the nucleic acid sequences encoding such polypeptides.

U.S. Pat. No. 5,182,261 discloses Modified TGF-alpha oligopeptides and pharmaceutical compositions thereof. This invention modifies TGF-alpha by replacement of an L amino acid residue with a D amino acid residue and pharmaceutical compositions thereof useful in wound healing, ulcer treatment or trauma.

Patent Publication no U.S. Pat. No. 5,102,870A discloses treatment and prevention of oral mucositis with growth factors. According to this invention, to prevent chemotherapy or radiotherapy induced oral mucositis in a mammal, an effective dose of a growth factor is administered. Longer contact with the mucosa! surface can be attained by selecting a suitable vehicle capable of coating mucosa.

U.S. Pat. No. 4,749,683 deals with the Inhibition of gastric acid secretion with TGF-alpha, which is an anti-secretory agent for the stomach.

Patent Publication no: U.S. Pat. No. 6,764,683 BI discloses Loop peptide and TGFα for stimulating stem cell proliferation and migration. According to this invention, is disclosed a novel genus of small peptides, much smaller than human TGFα. A novel peptide that is derived from a loop or “lollipop” region of transforming growth factor alpha (TGF-α) and is biologically active for causing stem cells to proliferate and migrate. The peptides are useful as pharmacologic agents for the same indications as full length TGFα polypeptide. Stimulates hematopoiesis in patients undergoing cytotoxic cancer chemotherapy and acts as a cytoprotective agent to protect a patient undergoing cancer cytotoxic therapy from gastrointestinal (GI) side effects, such as mucositis and otherwise support the barrier function of the GI tract when it is harmed by cytotoxic therapy.

Patent Publication no. U.S. Pat. No. 5,240,912 A (Application no: —U.S. Ser. No. 07/803,723) relates to Transforming growth factor (TGF) peptides, applied in both the cell growth field and in the detection and treatment of cancer and other proliferative diseases. TGF polypeptides, oligopeptides and antibodies raised to these polypeptides also have application in the detection and treatment of bone-loss diseases, such as osteoporosis, hypocalcemia and bone resorption.

Patent publication no.: U.S. Pat. No. 4,874,746 A (Application no.: U.S. Ser. No. 07/136,399) relates to wound healing composition of TGF-alpha and PDGF and healing an external wound or regenerating bone of a mammal by administering to the mammal a composition containing purified platelet-derived growth factor and purified transforming growth factor alpha. The composition of the invention provides a fast, effective method for healing external wounds of mammals, e.g., bed sores, lacerations and burns. The composition enhances connective tissue formation compared to natural healing (i.e. no exogenous agents added) or pure PDGF or TGF-α alone. Unlike pure PDGF alone, the composition promotes a significant increase in both new connective tissue and epithelial tissue. The epithelial layer obtained is thicker than that created by natural healing or by TGF-α alone, and contains more epithelial projections connecting it to the new connective tissue; it is thus more firmly bound and protective.

Post-injury skin tissue responses which include, epithelial cell proliferation and connective tissue remodeling. These responses are actively regulated by a set of growth factors. In chronic wounds, the cell proliferation is slowed down due to limited cell-cell contacts in the wound healing sites, which reduces the production of growth factors to a great extent. This results in the slow healing process as well as scar formation. Providing growth factors in the wound sites found to enhance the cell response to proliferate and often prevents scar formation. The major hinderance in the process is the cost of development of the growth factors and stable release of these peptides to the wound area.

### OBJECTS OF THE PRESENT INVENTION

It is therefore an object of this invention to design a Recombinant Transforming Growth Factor Alpha (TGFα) for various wound healing applications.

Another object of this invention is to provide the process for the preparation thereof of recombinant TGFα.

Another object of this invention is to provide a Recombinant Transforming Growth Factor Alpha (TGFα) using prokaryotic expression system, which drastically reduces the cost.

Yet another object of this invention is to provide a Recombinant Transforming Growth Factor Alpha (TGFα), which when delivered using an alginate scaffold matrix in the wound site, can heal the chronic wounds in a shorter period by remodeling the epithelial tissues with minimal scar formation.

Still another object of this invention is to provide a Recombinant Transforming Growth Factor Alpha (TGFα), which down regulates the wound healing pathway without any delay in time.

Further object of this invention is to provide a Recombinant Transforming Growth Factor Alpha (TGFα), which can be used as an alternative for skin grafting cosmetic surgery since the growth factor application can regrow the skin tissues back.

Yet further objective of the present invention is to disclose the efficacy of TGFα in wound healing.

Still further object of this invention is to provide a Recombinant Transforming Growth Factor Alpha (TGFα) and to deliver the growth factors to a suitable scaffold which suits the skin.

These and other objects and advantages of the invention will be apparent from the ensuing description.

### SUMMARY OF THE INVENTION

In one aspect of the invention, the present invention discloses and claims a recombinant transforming growth factor alpha (TGF α), having 51 amino acids, carved out of a 160 amino acid long TGF α, formed after post-translational modification of said 160 amino acid long TGF α, consisting of Sequence ID No.: I for wound healing applications.

In another aspect of the invention, the present invention discloses primers for amplifying TGF α consisting of Sequence ID Nos.: 2 and 3, wherein sequence ID No.: 2 is forward primer, and the sequence ID No.: 3 is reserve primer for TGF α.

In yet another aspect of the present invention, it discloses a process of preparation of a vector comprising essentially of Sequence ID No.: 1, said process comprising the steps of multiplying the desired gene fragment through polymerase chain reaction (PCR) using primers of Sequence ID No.: 2 and 3; eluting and digesting the PCR amplified product with pstl restriction endonuclease; sub-cloning the gene in pBS vector at position of restriction endonuclease BamHl/Xbal; sub-cloning further the gene to the host vector pGEX4Tl Smal site; transforming, expressing and screening the resultant plasmid vector in a prokaryotic system; tagging the recombinant protein with glutathione-S-transferase (GST); and purifying the recombinant protein tagged with GST to 95-97% purity level, wherein the reaction mixture composed of forward primers and reverse primers wherein I μI of each may be used per reaction, 2 μI of 10 mM of dNTP, I μI of 25 mM magnesium chloride, 5 μI of 10× buffer, 2.5 μI taq polymerase, 2 μI required gene fragment of Sequence ID No.: 1, 35.5 μI nuclease free water, thus making a total volume of 10 μ1.

In still another aspect, the present invention discloses a pharmaceutical composition comprising essentially of Sequence ID No.: 1, along with additives, fillers and addendums.

In most important aspect of the present invention, it discloses a method of wound healing using the recombinant TGF α comprising application of a pharmaceutical composition.

### BRIEF DESCRIPTION OF ACCOMPANYING DRAWINGS

The illustrated embodiments of the subject matter will be best understood by reference to the drawings, wherein like parts are designated by like numerals throughout. The following description is intended only by way of example, and simply illustrates certain selected embodiments of devices, systems, and processes that are consistent with the subject matter as claimed herein, wherein:

FIG. 1 provides the details of plasmid pGEX-4T-1, its relevant sequences, and restriction sites, along with position of antibiotic resistance and all other related details.

FIG. 2 reveals the fraction of TGF α, wherein lanes: I-0^{th} hour TGF α; 2-TGF α supernatant; 3-TGF α Purified E3 fraction (100 ul-+20 ul); 4-Pre stained Marker; 5-TGF α Purified E4 fraction (100 ul-+20 ul); 6-pGEX4T1 Supernatant; 7-TGF α purified E3 Fraction (un-concentrated); 8-TGF α purified E4 Fraction.

FIG. 3 details the result of MTT Assay in bar diagram, wherein on the “y” axis the percentage (%) of metabolic stability is shown on a scale of 0-120, wherein from the reader's left to right the bars are arranged, and wherein, I. For 2.5 μg concentration of TGF α; II. For 0.25 μg concentration of TGF α; III. For 0.025 μg concentration of TGF α; IV. For 0.0025 μg concentration of TGF α; V. For cell control; VI. For Reagent Control; VII. For Negative Control and VIII. For PositiveControl.

FIG. 4C shows how wound healing is found in control groups on 7^{th}, 14^{th} and 21^{st} days.

FIG. 4T shows how efficiently TGF α may be employed for wound healing in treated groups as against control groups on 7^{th}, 14^{th} and 21^{st} days.

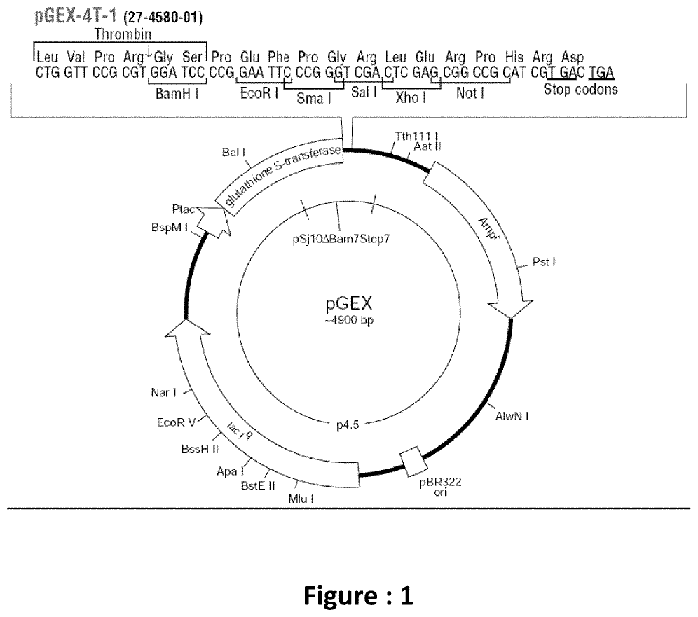

### DETAILED DESCRIPTION OF THE INVENTION

At the very outset of the detailed description, it may be understood that the ensuing description only illustrates a form of this invention. However, such a form is only exemplary embodiment, and without intending to imply any limitation on the scope of this invention. Accordingly, the description is to be understood as an exemplary embodiment and teaching of invention and not intended to be taken restrictively. Throughout the description and claims of this specification, the phrases “comprise” and “contain” and variations of them mean “including but not limited to”, and are not intended to exclude other moieties, additives, components, integers or steps. Thus, the singular encompasses the plural unless the context otherwise requires. Wherever there is an indefinite article used, the specification is to be understood as contemplating plurality as well as singularity, unless the context requires otherwise. Thus, the terms “comprises”, “comprising”, or any other variations thereof used in the disclosure, are intended to cover a non-exclusive inclusion, such that a device, system, assembly that comprises a list of components does not include—only those components but may include other components not expressly listed or inherent to such system, or assembly, or device.

In other words, one or more elements in a system or device proceeded by “comprises . . . a” does not, without more constraints, preclude the existence of other elements or additional elements in the system, apparatus or device.

Features, integers, characteristics, compounds, chemical moieties or groups described in conjunction with an aspect, embodiment or example of the invention are to be understood to be applicable to any other aspect, embodiment or example described herein unless incompatible therewith. All the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification including any accompanying claims, abstract and drawings or any parts thereof, or to any novel one, or any novel combination, of the steps of any method or process so disclosed.

The reader's attention is directed to all papers and documents which are filed concurrently with or before this specification in connection with this application and which are open to public inspection with this specification, and the contents of all such papers and documents are incorporated herein by reference. Post filing patents, original peer reviewed research paper shall be published.

The following descriptions of embodiments and examples are offered by way of illustration and not by way of limitation.

Unless contraindicated or noted otherwise, throughout this specification, the terms “a” and “an” mean one or more, and the term “or” means and/or. As used in the description herein and throughout the claims that follow, the meaning of “a.” “an,” and “the” includes plural reference unless the context clearly dictates otherwise. Also, as used in the description herein, the meaning of “in” includes “in” and “on” unless the context clearly dictates otherwise.

It may especially be noted that no animals have been harmed intentionally or unintentionally for the purposes of this invention. The animals used for the purpose of confirming the safety and efficacy of the present invention, were all treated with utmost care, and the smallest possible wound is made in a controlled manner following the treatment of the same with the outcome of the present invention.

Thus, according to this invention is provided a Recombinant Transforming Growth Factor Alpha (TGFα) for various wound healing applications.

Thus, according to another aspect of the same invention, there is provided a process of preparation thereof.

The present invention as accordance to the subject matter of concern, clones the 51 amino acid long active peptide sequence part of TGFα gene, i.e. Sequence ID No.: 1, and expresses in a standard expression system including but not limited to a prokaryotic expression system.

The strategy, therefore, is to clone the desired gene sequence coding for the mature peptide of TGFα, and to specifically identify and synthesize the most active part of the same following a post-translational modification of the cell.

The preferred embodiment of the present invention consists of expressing in prokaryotic expression system since said system expresses functionally active peptide, offers ease to express and allows purification of the growth factors in bulk, thus working positively on the cost front.

The preferred embodiment thus opens the possibility to exploits the crux of the present invention in wound dressing materials, designed for including but not limited to acute or chronic wounds, especially in chronic wounds.

In accordance with this invention, the active peptide of TGF α gene sequence is designed, PCR amplified and cloned into ampicillin resistant recombinant vector with a Glutathione S-Transferase tag and expressed in prokaryotic host.

For that the gene sequence of TGF α is designed, that codes for the functionally active peptide, having the sequence which targets specifically the wound healing signaling pathway. Since the mature peptide has been designed, it can directly bind to the receptors in the wound cells without the requirement of post translational modification.

The gene may be cloned and expressed in prokaryotic expression system including but not limited to bacterium. The prokaryotic expression system renders the invention cost effective. Since the recombinant protein is expressed in a bacterium, the vector with Glutathione S-Transferase tag has been used to purify the recombinant protein. This makes the action of purifying TGF α from the expressed bacterial system to a percentage of 85-90.

In the process front, TGF alpha gene is initially made into double strand by running the reaction without primer and then adding the primers to the PCR mixture, and then amplified using polymerase chain reaction (PCR).

For the PCR, the reaction mixture composed of forward primers and reverse primers, dNTP, magnesium chloride, buffer in a specific concentration, taq polymerase, genomic material including but not limited to DNA or RNA, nuclease free water, preferably distilled or demineralized.

The PCR amplified product is gel eluted and digested with pstl restriction endonuclease to confirm the product; upon digestion two bands are formed at 2 distinct position based on the molecular weight, something is determined by the number of base pairs. The PCR product is cloned to TA cloning vector for 3′-dA overhangs. The gene is then sub cloned to pBS vector at position of restriction endonuclease including BamHl/Xbal position. Then the gene is sub cloned to the host vector pGEX4Tl Smal site. The plasmid is then transformed, expressed and screened in a prokaryotic system.

The transformation protocol involves preparation of a competent cell following transformation. Preparation of competent cell includes the steps of inoculating a strain to a media and inoculating for certain period in a definite temperature while in rotational motion.

The culture thus obtained may be inoculated to a media, wherein incubation may be done till the OD reaches at 0.4. This may be followed by culturing to sterile polypropene tubes sealed by paraffin inoculate on ice for a few minutes followed by centrifugation. This is subjected to further processing by adding a calcium salt, cooling, resuspending, and final storing.

The transformation stage may start right here by incubating 100 μl of competent cells, and 1 μl of vector including but not limited to plasmid, phasmid, episome, or the likes, preferably a plasmid of suitable characterization, in a sterile tube. This may be followed by application of heat shock, followed by sudden cooling, adding competent media for a period while in a rotational motion.

This may be followed by cloning, wherein the plasmid insertion may be performed using certain restriction endonucleases either standalone or in combination, wherein the cleaving yield expected result as evidenced through the band in gel with respect to the respective molecular weights.

This may be followed by protein isolation using IPTG induction protocol, wherein an amount of culture of TGFα may be inoculated to media with required amount of antibiotic resistance and incubating the same at a definite temperature at a specific rotational motion in centrifugation. This may be followed by centrifuging the culture further for a certain time at a definite temperature, which yields the pallet, which are further resuspended using buffer.

This may be followed by adding lysozyme to the tubes, vertexing it well and keeping in ice followed by adding an amount of 0.2% Triton to each tube, and passing the solution through 5 ml syringe, keeping the tubes, and centrifuging further at a definite speed for a definite time at a definite temperature, following keeping the supernatant and adding DTT.

The isolated crude protein includes the bacterial as well as our protein of interest having 32 KDa was separated by Glutathione S transferase (GST) column chromatography. An affinity chromatography procedure was done with a glutathione Sepharose 4B column. This column matrix helps in binding GST Fusion proteins, which upon on contact with glutathione reduced elution buffer decrease its affinity towards Sepharose 4B and bind off from the stationary phase. The purified proteins may be qualitatively analyzed using SDS PAGE. The purity of the protein is measured as—85-90%.

The protein bulk purification protocol consists of pre equilibrating the column using IX Phosphate Buffer Saline (PBS)—10 bed volume, clarifying the crude protein sample with filter syringe and passing it through the column. Between each sample application the column may be washed with IX PBS. The elution of column with 6 ml of reduced glutathione buffer, collecting and storing of elution fractions, and reequilibration with 20 ml of IX PBS.

The recombinant TGF α is found to enhance the healing of chronic wounds, initiate cell proliferation in rabbit wounds within 7 days, and tissue regeneration and formation of epithelial layer on 14^{th} day as noted in in vivo model, especially animal models, more specifically on rabbit models. Prior testing in said in vivo model, an in vitro testing for the metabolic activity has been performed through executing MTT assay in a specific cell line.

On the safety side, recombinant TGF α passed the skin irritation test, sterility test and cytotoxicity tests, and found to be suitable for enhancing the healing of chronic wounds.

The present formulation is further clarified by giving the following exhibits. It must, however, be understood that these exhibits are only illustrative in nature and should not be taken as limitations to the capacity of the invention. Several amendments and improvements to the disclosed segments will be obvious to those skilled in the art. Thus, these amendments and improvements may be made without deviating from the scope of the invention.

### Example 1

Primer Designing:

The active peptide of TGF α gene sequence is designed, PCR amplified and cloned into ampicillin resistant (amp R) recombinant vector with a Glutathione S-Transferase tag and expressed in prokaryotic host. The complete sequence of the TGF isoform A, comprising 160 amino acids, wherein the gene sequence for expressing the mature peptide comprises 51 amino acids under Sequence ID No.: 1 is cloned with novel primers designed under Sequence ID No.: 2 and 3. The invention thus lies in craving out of the 51 peptides of interest from the 160 amino acid long sequence, and further multiplying it for human benefits.

As the principle aim of the invention is to develop a functionally active TGF-α peptide for wound healing application, the gene sequence of TGF alpha coding for the functionally active peptide, is designed, having the sequence which targets specifically the wound healing signaling pathway. Since the mature peptide has been designed, it can directly bind to the receptors in the wound cells without the requirement of post translational modification. And the gene is cloned and expressed in prokaryotic expression system. The prokaryotic expression system makes the invention cost effective. Since the recombinant protein is expressed in a bacterium, the vector with Glutathione S-Transferase tag has been used to purify the recombinant protein. This makes the action of purifying TGF alpha from the expressed bacterial system to a percentage of 85-90%.

The TGF alpha gene with 165 bp is designed and synthesized using two gene fragments; forward strand and reverse strand with 95 bp where the last 25 bp is the overlapping region. The TGF alpha gene is initially made into double strand by running the reaction for 6 minutes (first window) without primer and then the primers are added to the PCR mixture and then amplified using polymerase chain reaction. The novel primers so designed are disclosed categorically.

| Primers | Sequences |
|---|---|
| Forward | 5′-GAATTCGTGGTGTCCCCATTTTAATGA |
| SEQ. ID. NO.: 2 | CTGCC-3′ |
| Reverse | 5′-GAATTCTCAGGCCAGGAGGTCCGCA-3′ |
| SEQ. ID. NO.: 3 | |

### Example 2

Polymerase Chain Reaction (PCR) Using Novel Primers:

For the PCR, the reaction mixture composed of forward primers and reverse primers wherein 1 μl of each may be used per reaction, 2 μl of 10 mM of dNTP, 1 μl of 25 mM magnesium chloride, 5 μl of 10× buffer, 2.5 μl taq polymerase, genomic material including but not limited to DNA or RNA, preferably 2 μl DNA, 35.5 μl nuclease free water, thus making a total volume of 10 μl. It may be noted that all these ingredients for the reaction are procured and purchased from vendors supplying to the institute, where the research has been undertaken.

The PCR amplified product was gel eluted and digested with pstl restriction endonuclease to confirm the product; upon digestion two bands were formed at 2 distinct position based on the molecular weight, something is determined by the number of base pairs. The PCR product was cloned to TA cloning vector for 3′-dA overhangs. The gene was then sub cloned to pBS vector at position of restriction endonuclease BamHl/Xbal. Then the gene was sub cloned to the host vector pGEX4Tl Smal site. The plasmid was then transformed, expressed and screened in a prokaryotic system.

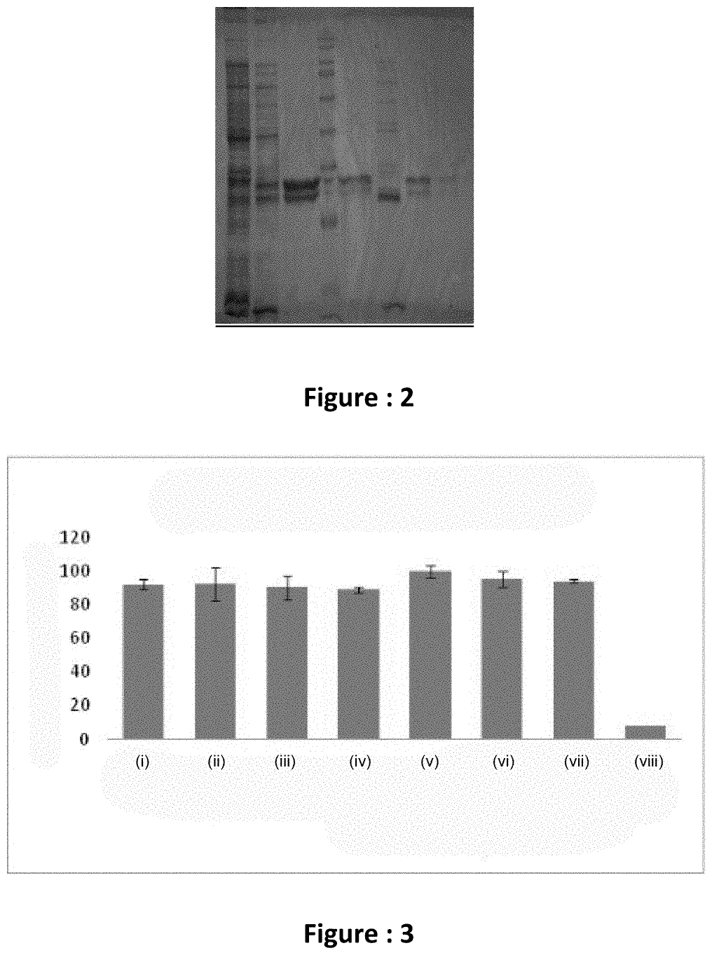

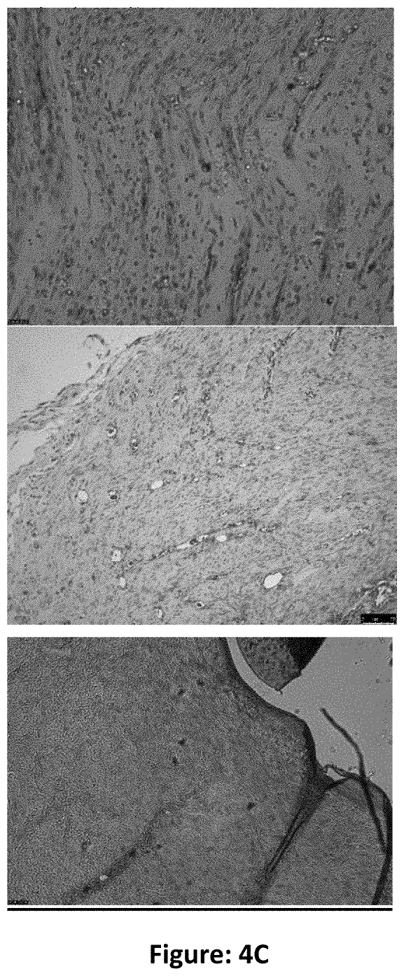

The PCR product was cloned to TA cloning vector pTZ. The gene was then sub-cloned to pBS vector at BamHl/Xbal sites. Then the gene was further sub-cloned to the host vector pGEX4Tl Smal site (Vector from Stratagene, USA), to be in the correct reading frame and to express as a glutathione-S-transferase (GST) tagged recombinant protein (FIG. 2). The plasmid was transformed, and recombinant protein was expressed in a prokaryotic system. Using GST affinity column chromatography, the recombinant protein tagged with GST was purified to 95-97% purity level as revealed under FIG. 3.

### Example 3

Preparation of Competent Cell for Transformation:

The transformation protocol involves preparation of a competent cell following transformation. Preparation of competent cell for day 1 includes the steps of inoculating XL1 blue strain to 5 ml LB media and inoculating overnight at 37° C. while keeping in 200 rpm.

2 ml of the culture thus obtained from day 1 may be inoculated 100 LB media, wherein incubation at 37° C. while keeping in 200 rpm, may be done till the OD reaches at 0.4. This may be followed by culturing to 50 ml of sterile polypropene tubes sealed by paraffin inoculate on ice for 5 minutes followed by centrifugation at 4500 rpm at 4° C. for 10 minutes. This is subjected to further processing by adding 200 mM calcium chloride solution while resuspending on 5 ml ice, cooling further on ice for 20 minutes, resuspending the resultant pellets and adding 15 ml of 80 mM calcium chloride, and final storing in fridge.

### Example 4

Transformation:

The transformation stage may start right here by incubating 100 μl of competent cells, and 1 μl of vector including but not limited to plasmid, phasmid, episome, or the likes, preferably a plasmid, in a sterile tube for 30 minutes on ice. This may be followed by application of heat shock at 42° C. for 90 seconds, followed by sudden cooling by placing on ice, adding 500 μl of LB media for a period of 45 minutes at 37° C. while kept at 350 rpm.

This may be followed by cloning confirmed by blue white selection, wherein the plasmid insertion may be performed using certain restriction endonucleases either standalone or in combination, wherein Pstl digestion may yield 2 bands at 4081 and 1053 in gel with respect to the respective molecular weights as sated.

### Example 5

Protein Isolation Using IPTG Induction Protocol:

The transformation as disclosed may be followed by protein isolation using IPTG induction protocol, which triggers the lac operon gene in the pGEX4Tl vector, wherein I ml of culture of TGFα may be inoculated to I 00 ml of LB media with required amount of antibiotic resistance i.e. ampR and incubating the same at at 37° C. at 200 rpm in centrifugation. When the OD value reaches 0.6, 0.1 mM IPTG may be added, followed by incubation for 3 hours at 200 rpm at 37° C. The culture is then transferred into sterile centrifuge tubes and centrifuged for 14 minutes at 500 g at 4° C. This may be followed by isolating the pellets and resuspending the same using 1 ml 1×PBS.

This may be followed by adding 1 mg/ml lysozyme to the tubes, vertexing it well and keeping in ice for 30 minutes followed by adding 10 ml of 0.2% Triton to each tube, and passing the solution through 5 ml syringe, keeping the tubes at 4° C. for 30 minutes, and centrifuging further at 3000 g for 30 min at 4° C., following the addition of 1 mM DTT.

The isolated crude protein includes the bacterial as well as the protein of interest having 32 KDa was separated by Glutathione S transferase (GST) column chromatography. An affinity chromatography procedure was done with a glutathione Sepharose 4B column. This column matrix helps in binding GST Fusion proteins, which upon on contact with glutathione reduced elution buffer decrease its affinity towards Sepharose 4B and bind off from the stationary phase. The purified proteins may be qualitatively analyzed using SDS PAGE. The purity of the protein is found to be—85-90%.

### Example 6

Protein Bulk Purification Protocol:

The protein bulk purification protocol consists of pre equilibrating the column using I×PBS—10 bed volume, clarifying the crude protein sample with 0.22 mm filter syringe and passing it through the column. Between each 2 ml sample application the column is washed with 2 ml of 1×PBS, elution of column with 6 ml of reduced glutathione buffer, collecting and storing of elution fractions and storing. The column may be re-equilibrated with 20 ml of 1×PBS.

### Example 7

MTT Assay:

The MTT assay is carried out in L929 cells (ATCC, USA). The following concentrations of purified recombinant TGF α were used: 0.0025 μg/ml, 0.025 μg/ml, 0.25 μg/ml and 2.5 μg/ml; the cell viability was found to be 92.16%. 92.26%, 90.30% and 88.87% under these concentrations respectively. Positive control, reagent control and negative control showed 8.14%, 95.16% and 94.25% metabolic activity respectively. The results obtained are depicted in FIG. 3.

### Example 8

Study for Efficacy and Safety:

The antimicrobial test has been performed on test material, meeting the requirements for dermal applications. The declaration for skin irritation test is hereby made as the recombinant TGFα induced a total mean score of ‘0.55’ in test material and ‘◯’ in solvent following intra-dermal injection. No intra-cutaneous reactions such as erythema and edema has been indicated in the test.

For this study, 23 rabbits (New Zealand White) are taken and kept in a well-fed, healthy and aseptic condition to ensure the perfect outcome of the experimentation.

Nine randomly selected rabbits (New Zealand White) are taken and a 4×4 cm incision was made right on the back of the animals. Following the application of Anesthetic Ketamine (Aneket, Neon Laboratories, India). This has been followed by application of recombinant TGFα to the “treated” group, and application of placebo, wherein except the growth factor, all the other conditions of “treated” group is followed.

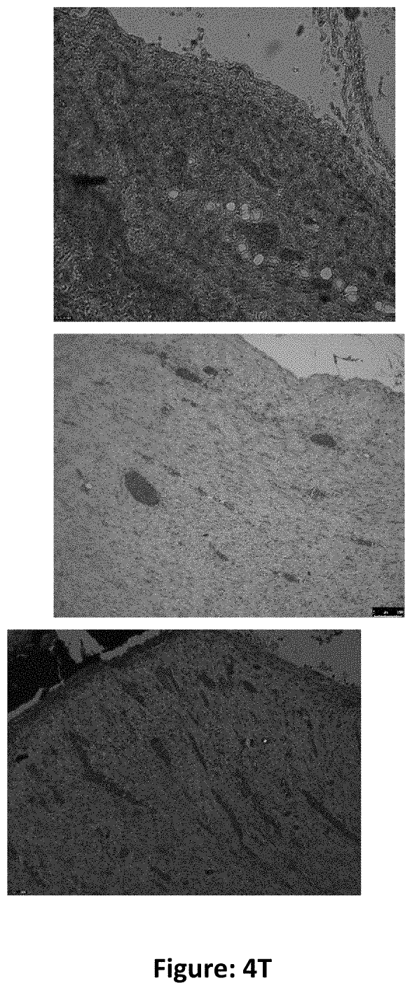

The recombinant transforming growth factor alpha (TGF α) found to enhance the healing of chronic wounds. It is found to initiate cell proliferation in rabbit wounds within 7 days. On 14^{th} day tissue regeneration and formation of epithelial layer was noted in rabbit models. Thus, the recombinant growth factor TGF α found to be suitable for enhancing the healing of chronic wounds. The healing wound showed higher number of vasculature and almost no inflammatory response, as evidenced from the comparative FIGS. 4C and 4T.

FIGS. 4C and 4T shows how efficiently TGF α may be employed for wound healing as against control groups on 7^{th}, 14^{th} and 21^{st} days.

In sum, the present invention, although works with certain peptides, and designs certain DNA sequences, in no condition, these may be considered as “Biological Material” as per the definition of the Biodiversity Act, saying “Biological resources as plants, animals and micro-organisms or parts thereof, their genetic material and by-products (excluding value added products) with actual or potential use or value, but does not include human genetic material”, therefore, this present invention may have nothing to do with the living world except for working with certain proteins, which are merely biochemical entities, easily synthesized in laboratory conditions. Neither the proteins are parts of genetic material, nor there is any genetic sequence in designed primers. All the source and geographical origins are, however, categorically disclosed.

Now, the crux of the invention is claimed implicitly and explicitly through the following claims.

Each of the appended claims defines a separate invention, which for infringement purposes is recognized as including equivalents to the various elements or limitations specified in the claims. Depending on the context, all references below to the “invention” may in some cases refer to certain specific embodiments only. In other cases, it will be recognized that references to the “invention” will refer to subject matter recited in one or more, but not necessarily all, of the claims.

Groupings of alternative elements or embodiments of the invention disclosed herein are not to be construed as limitations. Each group member can be referred to a claimed individually or in any combination with other members of the group or other elements found herein. One or more members of a group can be included in or deleted from, a group for reasons of convenience and/or patentability. When any such inclusion or deletion occurs, the specification is herein deemed to contain the group as modified thus fulfilling the written description of all groups used in the appended claims.

We claim:

1. A recombinant transforming growth factor alpha (TGF α) consisting of Sequence ID No.: 1 for wound healing applications.

2. The recombinant TGF α under Sequence ID No.: 1 as claimed in claim 1, wherein said 51 amino acid long sequence is carved out from the 160 amino acid long TGF α.

3. Primers for amplifying TGF α consisting of Sequence ID Nos.: 2 and 3.

4. The primers as claimed in claim 3, wherein Sequence ID No.: 2 is forward primer, and the Sequence ID No.: 3 is reserve primer for the recombinant TGF α under Sequence ID No.: 1.

5. A process of preparation of a vector comprising essentially of Sequence ID No.: 1 as claimed in claim 1, said process comprising the steps of:
a) multiplying the desired gene fragment through polymerase chain reaction (PCR) using primers of Sequence ID No.: 2 and 3;
b) eluting and digesting the PCR amplified product with pst1 restriction endonuclease;
c) sub-cloning the gene to pBS vector at position of restriction endonuclease BamH1/Xba1;
d) sub-cloning further the gene to the host vector pGEX4T1 Sma1 site;
e) transforming, expressing and screening the resultant plasmid vector in a prokaryotic system;
f) tagging the recombinant protein with glutathione-S-transferase (GST); and
g) purifying the recombinant protein tagged with GST to 95-97% purity level.

6. The process as claimed in claim 5, wherein the reaction mixture composed of forward primers and reverse primers wherein 1 μl of each may be used per reaction, 2 μl of 10 mM of dNTP, 1 μl of 25 mM magnesium chloride, 5 μl of 10× buffer, 2.5 μl taq polymerase, 2 μl required gene fragment of Sequence ID No.: 1, 35.5 μl nuclease free water, thus making a total volume of 10 μl.

7. A pharmaceutical composition comprising essentially of Sequence ID No.: 1, along with additives, fillers and addendums.

8. A method of wound healing using the recombinant TGF α comprising application of a pharmaceutical composition as claimed in claim 7.
